(19) United States
(12) Patent Application Publication
(10) Pub. No.: US 2013/0012531 A1
(43) Pub. Date: Jan. 10, 2013

(54) TREATMENT METHOD

(76) Inventors: Andrew G King, King of Prussia, PA (US); Albert B. Suttle, Research Triangle Park, NC (US); Valeriu Damian-Iordache, King of Prussia, PA (US); Megan M Mclaughlin, King of Prussia, PA (US)

(21) Appl. No.: 13/518,407

(22) PCT Filed: Jan. 5, 2011
(86) PCT No.: PCT/US11/20231

§ 371 Date: Sep. 27, 2012

Related U.S. Application Data
(60) Provisional application No. 61/292,747, filed on Jan. 6, 2010.

(51) Int. Cl. A61K 31/506; A61K 31/404; A61P 27/02; A61K 31/44

(52) U.S. Cl. 514/275; 514/350; 514/414

(57) ABSTRACT

The present invention is directed to methods of treating disorders of ocular angiogenesis or vascular leakage in a patient by administration of suitable inhibitors, including pazopanib or pharmaceutically acceptable salts or hydrates thereof.

### FIELD OF THE INVENTION

The present invention relates to methods of treating disorders of ocular angiogenesis or vascular leakage in a mammal. The methods comprise administering pyrimidine derivatives, benzodiazepinyl derivatives, and pharmaceutical compositions containing the same.

### BACKGROUND OF THE INVENTION

Neovascularization, also called angiogenesis, is the process of forming new blood vessels. Neovascularization occurs during normal development, and also plays an important role in wound healing following injury to a tissue. However, neovascularization has also been implicated as an important cause of a number of pathological states including, for example, cancer, rheumatoid arthritis, atherosclerosis, psoriasis, and diseases of the eye.

An eye disorder in which neovascularization plays a role is age-related macular degeneration (AMD), which is the major cause of severe visual loss in the elderly. The vision loss in AMD results from choroidal neovascularization (CNV). The neovascularization originates front choroidal blood vessels and grows through Bruch's membrane, usually at multiple sites, into the sub-retinal pigmented epithelial space and/or the retina (see, for example, Campochiaro et al. (1999) Mol. Vis. 5:34). Leakage and bleeding from these new blood vessels results in vision loss.

### SUMMARY OF THE INVENTION

In an aspect of the present invention, a method of treating a disorder of ocular angiogenesis or vascular leakage in a patient suffering from such condition includes orally administering to the patient between 1 and 50 mg of a suitable inhibitor.

In another aspect of the present invention, a method of treating a disorder of ocular angiogenesis or vascular leakage in a patient suffering from such condition includes orally administering to the patient between 1 and 50 mg of a compound of formula (I):

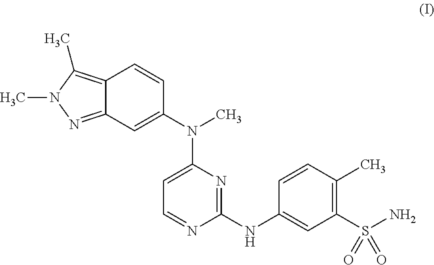 [SMILES: Cc1ccc(Nc2nccc(N(C)c3ccc4c(C)n(C)nc4c3)n2)cc1S(N)(=O)=O]

or a pharmaceutically acceptable salt or hydrate thereof.

In still another aspect according to the present invention, the use of a suitable inhibitor in the manufacture of a medicament containing between 1 and 50 mg of the suitable inhibitor for the treatment of a disorder of ocular angiogenesis or vascular leakage in a patient in need thereof is provided.

In yet another aspect according to the present invention, there is provided between 1 and 50 mg of a suitable inhibitor for use in the treatment of a disorder of ocular angiogenesis or vascular leakage in a patient in need thereof.

### BRIEF DESCRIPTION OF THE DRAWINGS

FIG. 1A illustrates representative fluorescein angiograms with a pazopanib-induced change of the CNV leakage in experimental CNV;

FIG. 1B illustrates analysis of fluorescein leakage areas for treatment with vehicle and pazopanib. Changes were determined using digital image analysis (*** p<0.005);

FIG. 2A illustrates representative light micrographs of hematoxylin and eosin-stained areas comprising neovascular lesions (encircled) on post-laser day 14;

FIG. 2B illustrates averaged lesion areas from eyes treated with the vehicle or pazopanib (***p<0.005; n=6);

FIG. 2C illustrates averaged lesion areas from eyes when the fellow eye was treated with the vehicle of pazopanib;

FIG. 3A illustrates single dose plasma kinetics in Brown Norway rats (composite data from n=3 rats per point); and

FIG. 3B illustrates plasma and eye cup (sclera/choroid/retina) pazopanib content 5 hours after the third eye drop administered over 24 hours. OS—left treated eye, OD—fellow non-treated eye (n=3 rats per point);

### DETAILED DESCRIPTION OF THE INVENTION

The invention provides methods for treating a disorder of ocular angiogenesis or vascular leakage, such as age-related macular degeneration. As used herein, “treatment” means any manner in which one or more symptoms associated with the disorder are beneficially altered. Accordingly, the term includes healing or amelioration of a symptom or side effect of the disorder or a decrease in the rate of advancement of the disorder.

As used herein, the term “suitable inhibitor” means an inhibitor that inhibits one or more of the following receptors: VEGFR1, VEGFR2, VEGFR3, PDGFRalpha, PDGFRbeta, c-kit, and/or FGFR.

As used herein, the term “therapeutically effective amount” means the amount of a therapeutic agent that is sufficient to treat, prevent and/or ameliorate one or more symptoms of the disorder.

According to embodiments of the present invention, a method of treating a disorder of ocular angiogenesis or vascular leakage in a patient suffering from such condition includes orally administering to the patient between 1 and 50 mg of a suitable inhibitor.

The suitable inhibitor can be various inhibitors that inhibit one or more of the following receptors: VEGFR1, VEGFR2, VEGFR3, PDGFRalpha, PDGFRbeta, c-kit, and/or FGFR including, but not limited to: a compound of formula (I) or a pharmaceutically acceptable salt or hydrate thereof, a compound of formula (I′) or a hydrate thereof, a complex of formula (I″), a compound of formula (II) or a pharmaceutically acceptable salt thereof, apatinib, sunitinib, sorafenib, bivanib, midostaurin (PKC412) (an inhibitor of FLT3, c-KIT, VEGFR-2, PDGFR and multiple isoforms of the serine/threonine protein kinase C (PkC), under development by Novartis), E-7050 (a C-met and VEGFR tyrosine kinase inhibitor, under development by Eisai), XL-184 a spectrum-selective kinase inhibitor that inhibits Met, Ret and VEGFR2, under development by Exelixis), XL-647 (an orally-available tyrosine kinase inhibitor under development by Exelixis that inhibits EGFR, HER2, VEGFR and EphB4), cediranib, linifanib, motesanib, RAF-265 (formerly CHIR-265) a B-Raf and VEGFR kinase inhibitor, under development by Novartis), tivozanib, TAK-593 (a VEGFR/PDGFR tyrosine kinase inhibitor, under development by Millennium (Takeda)), ARQ-197 (an ATP-independent inhibitor of c-Met under development by ArQule (Cyclis Pharmaceuticals before the acquisition)), OSI-930 (a c-kit and VEGFR-2 tyrosine kinase inhibitor under development by OSI Pharmaceuticals), DCC-2036 (a Bcr-abl inhibitor inhibits that also inhibits the Src-like kinases LYN, HCK and FGR, as well as the TIE2 and KDR kinases, under development by Deciphera Pharmaceuticals), MGCD-265 (an inhibitor of c-Met, VEGFR1, VEGFR2, VEGFR3, Tie-2 and Ron tyrosine receptor kinases, under development by MethylGene, in collaboration with ChemBridge Research Laboratories, CA, the US), PF-337210 (an inhibitor of VEGFR2, under development of Pfizer), BIBF-1120 (a VEGFR-2, PDGF and FGF kinase inhibitor, which also inhibits the src, lck and lyn tyrosine kinases, under development by Boehringer Ingelheim), ENMD-2076 (a kinase inhibitor that selectively targets aurora kinase A vs B, and also inhibits Flt3, c-kit, CSF1R and KDR (VEGFR2) as well as VEGFR3, PDGFR-alpha, FGFR1, FGFR2, EphA1 and src, under development by EntreMed), TG-100-801 (a VEGFR, Src, Yes, Lek, Lyn kinases and PDGFR-β inhibitor, under development by TargeGen), BMS-690514 (an inhibitor of EGFR, HER2, ErbB4 and VEGFR1-3, under development by Bristol-Myers Squibb), SSR-106462 (a TIE-2 inhibitor and VEGFR-2 tyrosine kinase inhibitor, under development by Sanofi-Aventis), BAY-73-4506 (a VEGFR, KIT, RET, FGFR and PDGFR kinase inhibitor, under development by Bayer), plitidepsin, axitinib, vandetinib, and nilotinib. The suitable inhibitors may be in the form of pharmaceutically acceptable salts and, in some cases, in the form of pharmaceutically acceptable solvates to the extent that such suitable inhibitors have been described in the art as being in a solvated form.

In some embodiments, the suitable inhibitor is pazopanib or a pharmaceutically acceptable salt or solvate thereof, such as a compound of formulae (I) or a pharmaceutically acceptable solvate thereof, a compound of formula (I′) or a solvate thereof, or a complex of formula (I″). In other embodiments, the suitable inhibitor is a compound of formula (II) or a pharmaceutically acceptable salt thereof. In still other embodiments, the suitable inhibitor is sorafenib or a pharmaceutically acceptable salt thereof, such as the tosylate salt. In still other embodiments, the suitable inhibitor is sunitinib or a pharmaceutically acceptable salt thereof, such as the malate salt.

According to embodiments of the present invention, a method of treating a disorder of ocular angiogenesis or vascular leakage in a patient suffering from such condition includes orally administering to the patient between 1 and 50 mg of a compound of formula (I):

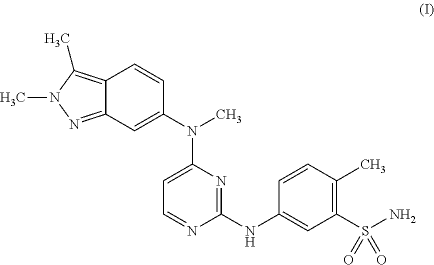 [SMILES: Cc1ccc(Nc2nccc(N(C)c3ccc4c(C)n(C)nc4c3)n2)cc1S(N)(=O)=O]

or a pharmaceutically acceptable salts or hydrates thereof.

The compound of formula (I) has the chemical name 5-[[4-[(2,3-dimethyl-2H-indazol-6-yl)methylamino]-2-pyrimidinyl]amino]-2-methylbenzenesulfonamide and the generic name pazopanib.

In certain embodiments, the salt of the compound of formula (I) is a hydrochloride salt. In a particular embodiment, the salt of the compound of formula (I) is a monohydrochloride salt as illustrated by formula (I′). The monohydrochloride salt of the compound of formula (I) has the chemical name 5-[[4-(2,3-dimethyl-2H-indazol-6-yl)methylamino]-2-pyrimidinyl]amino]-2-methylbenzenesulfonamide monohydrochloride.

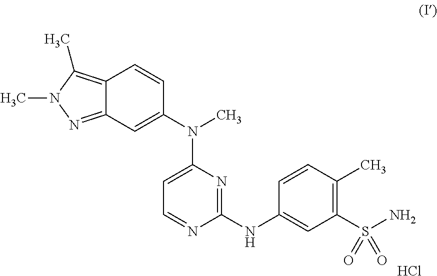 [SMILES: Cc1ccc(Nc2nccc(N(C)c3ccc4c(C)n(C)nc4c3)n2)cc1S(N)(=O)=O ; Cl]

In other embodiments, the salt of the compound of formula (I) is a monohydrochloride monohydrate solvate of the compound of formula (I). The monohydrochloride monohydrate solvate of the compound of formula (I) has the chemical name 5-({4-[(2,3-dimethyl-2H-indazol-6-yl)methylamino]-2-pyrimidinyl}amino)-2-methylbenzenesulfonamide monohydrochloride monohydrate, as illustrated in formula (I′).

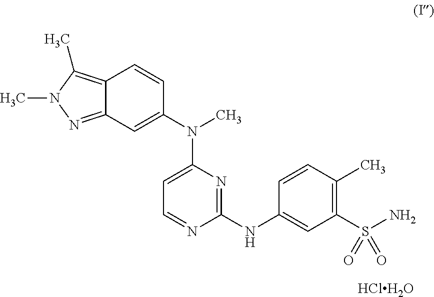

The free base, salts and hydrates of the compound of formula (I) may be prepared, for example, according to the procedures of International Patent Application No. PCT/US01/49367 filed Dec. 19, 2001, and published as WO 02/059110 on Aug. 1, 2002, and International Patent Application No. PCT/US03/19211 filed Jun. 17, 2003, and published as WO 03/106416 on Dec. 24, 2003.

According to embodiments of the present invention, a method of treating a disorder of ocular angiogenesis or vascular leakage in a patient suffering from such condition includes orally administering to the patient between 1 and 50 mg of a compound of formula (II):

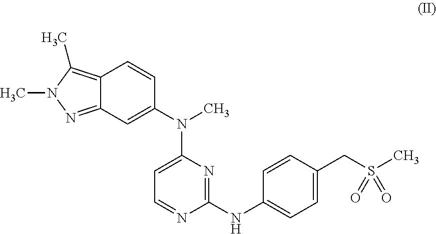

or a pharmaceutically acceptable salt thereof. The free base and salts of the compound of formula (II) may be prepared, for example, according to the procedures of International Patent Application No. PCT/US01/49367 filed Dec. 19, 2001, and published as WO 02/059110 on Aug. 1, 2002, and International Patent Application No. PCT/US03/19211 filed Jun. 17, 2003, and published as WO 03/106416 on Dec. 24, 2003.

As used herein, the term “pharmaceutically acceptable salts” may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate.

In embodiments of methods according to the invention, the disorder of ocular angiogenesis or vascular leakage can be edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

In one embodiment, the neovascular age-related macular degeneration is wet age-related macular degeneration. In another embodiment, the neovascular age-related macular degeneration is dry age-related macular degeneration and the patient is characterized as being at increased risk of developing wet age-related macular degeneration.

While it is possible that the suitable inhibitor may be administered as the raw chemical, it is also possible to present the active ingredient as a pharmaceutical composition. Accordingly, embodiments of the invention further provide pharmaceutical compositions, which include therapeutically effective amounts of the suitable inhibitor, and one or more pharmaceutically acceptable carriers, diluents, or excipients. The suitable inhibitor is as described above. In one embodiment, the suitable inhibitor is a compound of formula (I) or a pharmaceutically acceptable salt or hydrate thereof In another embodiment, the suitable inhibitor is a compound of formula (I′) or a hydrate thereof. In still another embodiment, the suitable inhibitor is a complex of formula (I″). In yet another embodiment, the suitable inhibitor is a compound of formula (II) or a pharmaceutically acceptable salt thereof. In still other embodiments, the suitable inhibitor is sorafenib or a pharmaceutically acceptable salt thereof, such as the tosylate salt. In still other embodiments, the suitable inhibitor is sunitinib or a pharmaceutically acceptable salt thereof, such as the malate salt. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing the suitable inhibitor with one or more pharmaceutically acceptable carriers, diluents or excipients.

Pharmaceutical formulations may be presented in unit dose forms containing a predetermined amount of active ingredient per unit dose. In certain embodiments, the unit dosage formulations are those containing a dose to be administered as frequently as daily or sub-dose or an appropriate fraction thereof of an active ingredient. Furthermore, such pharmaceutical formulations may be prepared by any of the methods well known in the pharmacy art.

Pharmaceutical formulations may be adapted for oral administration. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The suitable inhibitor can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The suitable inhibitor may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

It should be understood that in addition to the ingredients particularly mentioned above, the formulations may include other agents conventional in the art having regard to the type of formulation in question, for example those suitable for oral administration may include flavouring agents.

According to the methods of the invention, a suitable inhibitor is administered or prescribed to a patient. The amount of administered or prescribed compound will depend upon a number of factors including, for example, the age and weight of the patient, the precise condition requiring treatment, the severity of the condition, and the nature of the formulation. Ultimately, the amount will be at the discretion of the attendant physician.

In some embodiments of the methods of the invention, the total amount of the suitable inhibitor administered or prescribed to be administered as frequently as daily can be from a lower limit of 1, 2, 3, 4, 5, 6, 7, 8, 9, 10, 11, 12, 13, 14, 15, 16, 17, 18, 19, or 20 mg to an upper limit of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg. The suitable inhibitor is as described above. In one embodiment, the suitable inhibitor is a compound of formula (I) or a pharmaceutically acceptable salt or hydrate thereof. In another embodiment, the suitable inhibitor is a compound of formula (I′) or a hydrate thereof. In still another embodiment, the suitable inhibitor is a complex of formula (I″). In yet another embodiment, the suitable inhibitor is a compound of formula (II) or a pharmaceutically acceptable salt thereof. In still other embodiments, the suitable inhibitor is sorafenib or a pharmaceutically acceptable salt thereof, such as the tosylate salt. In still other embodiments, the suitable inhibitor is sunitinib or a pharmaceutically acceptable salt thereof, such as the malate salt.

The methods of the present invention may also be employed in combination with other methods for the treatment of ocular neovascular disorders. In some embodiments, the methods of the invention encompass a combination therapy in which a suitable inhibitor is administered in conjunction with one or more additional therapeutic agents for the treatment of neovascular disorders, which therapeutic agents can, themselves be suitable inhibitors as described herein. In one embodiment, a suitable inhibitor used in the combination is a compound of formula (I) or a pharmaceutically acceptable salt or hydrate thereof. In another embodiment, a suitable inhibitor used in the combination is a compound of formula (I′) or a hydrate thereof. In still another embodiment, a suitable inhibitor used in the combination is a complex of formula (I″). In yet another embodiment, a suitable inhibitor used in the combination is a compound of formula (II) or a pharmaceutically acceptable salt thereof. In still another embodiment, a suitable inhibitor used in the combination is sorafenib or a pharmaceutically acceptable salt thereof, such as the tosylate salt. In still another embodiment, a suitable inhibitor used in the combination is sunitinib or a pharmaceutically acceptable salt thereof, such as the malate salt. Non-limiting examples of additional therapeutic agents that may be used in a combination therapy include pegaptanib, ranibizumab, bevacizumab, midostaurin, nepafenac, integrin receptor antagonists (including vitronectin receptor agonists), and any of the various suitable inhibitors described herein. See, for example, Takahashi et al, (2003) Invest. Ophthalmol. Vis. Sci. 44: 409-15, Campochiaro et al. (2004) Invest, Ophthalmol. Vis, Sci. 45:922-31, van Wijngaarden et al. (2005) JAMA 293:1509-13, U.S. Pat. No. 6,825,188 to Callahan et al., and U.S. Pat. No. 6,881,736 to Manley et al.; each of which is herein incorporated by reference for their teachings regarding these compounds.

Where a combination therapy is employed, the therapeutic agents may be administered together or separately. The same means for administration may be used for more than one therapeutic agent of the combination therapy; alternatively, different therapeutic agents of the combination therapy may be administered by different means. When the therapeutic agents are administered separately, they may be administered simultaneously or sequentially in any order, both close and remote in time. The amounts of the suitable inhibitor and/or the other pharmaceutically active agent or agents and the relative timings of administration will be selected in order to achieve the desired combined therapeutic effect.

The following examples are intended for illustration only and are not intended to limit the scope of the invention in any way.

### EXAMPLES

The free base, salts and hydrates of pazopanib used in these examples may be prepared, for example, according to the procedures of International Patent Application No. PCT/US01/49367 filed Dec. 19, 2001, and published as WO 02/059110 on Aug. 1, 2002, and International Patent Application No. PCT/US03/19211 filed Jun. 17, 2003, and published as WO 03/106416 on Dec. 24, 2003.

### Biological Data:

### Reagents

Topical eye drops were formulated in a buffered 7% cyclodextrin solution containing 5 mg/ml pazopanib. Sodium fluorescein (10% w/v) was purchased from Alcon (Alcon Pharma, Freiburg, Germany). Endothelial cell basal medium (EBM) and endothelial cell growth medium (EGM) were obtained from Lonza, Verviers, Belgium. Hank's balanced salt solution (HBSS) and Ham's-F10 were from Invitrogen (Karlsruhe, Germany). All other chemicals were reagent-grade products obtained commercially from Sigma (Taufkirchen, Germany).

### Animals and Anaesthesia

Male Brown Norway rats (10-12 weeks of age, male and female weighing 170 to 360 g) were used throughout this study. The animals were treated according to ARVO Statement on the use of animals in ophthalmic and vision research, and all animal experiments were reviewed and approved by municipal and University Hospital animal care committees in Leipzig. The rats were anesthetized with intraperitoneal ketamine (100 mg/kg; Ratiopharm, Ulm, Germany) and xylazine (BayerVital, Leverkusen, Germany; 10 mg/kg). Topical application of tropicamide (5 mg/ml) and phenylephrine hydrochloride (Ankerpharm, Rudolstadt, Germany; 50 mg/ml) were instilled for mydriasis during laser photocoagulation and fluorescein angiography. Fourteen days after laser injury, rats were humanely euthanized using overdoses of carbon dioxide.

Induction of CNV in Rats and Treatment with Pazopanib

Animals were treated using laser photocoagulation-induced rupture of Bruch's membrane using a 545 nm dye laser (Coherent Argon Dye Laser #920, Coherent Medical Laser, Palo Alto, Calif.) attached to a slit lamp (Carl Zeiss, Oberkochen, Germany). A contact lens was used to retain corneal clarity through photocoagulation. The laser spots were placed separately using the following settings; 50-μm diameter, 0.1 second duration, and 180-mW intensity. To rupture Bruch's membrane, four to seven laser spots were applied between the major retinal vessels close to the optic disc. Pazopanib (approximately 30 μl of a 5 mg/ml sterile-filtered solution) was administered twice daily. Animals of the control group received a vehicle only.

### Fluorescence Angiography in Experimental CNV

Aiming to document treatment of CNV in its early stage, a treatment schedule was used as follows. Laser photocoagulation was carried out as described above, and pazopanib was applied twice a day topically from post laser day 6 until study end on post laser day 14. Coagulated lesions were first documented by angiography on day 7 post laser, and only rats with ocular CNV were included in the analysis. Sodium fluorescein was injected into tail vein of the anesthetized rats and fluorescein angiograms were obtained by means of a fundus camera (FD-31A, Topcon, Tokyo, Japan). On day 14, rats underwent a second angiography. Angiograms taken 100 to 140 seconds after injection were converted to digital images, and areas of fluorescein leakage with intensity as high as in major vessels were quantified in a masked fashion BY two of us (YY; XMY) using a computer software (NIH image, Research Service Branch, Bethesda, Md.). Differences in fluorescence were calculated by the following formula:

Area of fluorescein leakage on day 14×100%/area of fluorescein leakage on day 7.

### Histology and Immunohistochemistry

After the rats were euthanized on day 14, eyes were immediately dissected and fixed with 4% paraformaldehyde (dissolved in PBS). Five minutes later, the eyes were perforated at the equator and left overnight at 4° C. in the same solution. Subsequently, the eyes were divided into anterior and posterior segments with total removal of lens and vitreous body. Serial six-micrometer sections were prepared and either stained with hematoxylin and eosin (HE) or processed for immunohistochemistry. HE-stained sections were examined at 200× magnification using a light microscope (Axioplan 2; Carl Zeiss Meditec, Jena, Germany) and a digital color camera (AxioCam MRc5; Carl Zeiss Meditec). The maximal area of CNV complexes was estimated indirectly, by measuring the difference between the thickness from the outer border of the pigmented choroidal layer to the top of the CNV complex and the thickness of the intact, pigmented choroids adjacent to the lesion. Three to five serial sections from each CNV membrane were measured, and the highest value (representing the top of a given CNV complex) was stored. Digitized images were analyzed and measured with the concomitant image-analysis software (Axiovision; Carl Zeiss). Each lesion was encircled manually, and their area (in μm²) was calculated by the program.

Additionally, some sections were stained with a polyclonal goat anti-rat VEGF antibody (R&D Systems). Briefly, sections were washed using PBS-TD (PBS/1% dimethyl sulfoxide/0.3% Triton X-100) followed by quenching endogenous peroxidase activity in PBS/0.3% H₂O₂ for 5 min followed by washing in PBS. Subsequently, sections were blocked with PBS-TD/10% rabbit normal serum at 37° C. for 1 h and incubated with anti-VEGF (5 μg/ml in PBS/2% BSA) overnight. In negative control sections, the primary antibody was replaced by normal goat immunoglobulin (Ig)G. After washing three times with PBS-TD, the slices were incubated at room temperature with horseradish peroxidase-conjugated rabbit anti-goat IgG (Dianova, Hamburg, Germany; diluted 1:1000 in PBS/2% BSA) for 2 h. A buffered solution of 3,3′-diaminobenzidine (Vector Laboratories, Burlingame, Calif.) was used as a chromogen together with H₂O₂ in order to detect peroxidase activity. Sections were counterstained with Meyer's hematoxylin, washed in PBS and water and mounted. All slides were examined using a Zeiss Axioskop microscope equipped with a digital camera.

### Ocular Tissue Drug Concentration Procedures

Female Norway Brown rats were used during these studies. Rats were purchased from Charles River (Portage, Mich.). In two independent studies, Norway Brown rats received 30 μl pazopanib eye drops (5 mg/ml in buffered 7% cyclodextrin) for either 24 hours (3 total drops given every 8-12 hours), once daily for 5 days, or twice daily for 14 days.

### Tissue Collection Procedures

Rats were euthanized by CO₂ inhalation before enucleation and collection of plasma samples. Samples were frozen immediately over dry ice after collection and then stored at −80° C. The ocular tissues were processed through a dissection-pulverization-drug extraction process. Frozen eye sectioning was performed by the following steps. The preparation of rat eye cups was done by removing the anterior portion of the eye with a razor blade followed by removal of the lens and frozen vitreous with forceps. A sagittal section was made in the eye cup before collection of the retina/choroid tissue by scraping the exposed sclera with the round end of a spatula until the pigmented tissue was completely removed from the scleral tissue. The collected tissues were pulverized under liquid nitrogen. Frozen tissue was carefully placed in a liquid nitrogen primed BioPulverizer (Biospec Products Inc, Bartlesville, Okla.). Following pulverization the tissue powder was removed from the pulverizer and transferred into a polypropylene tube. Extraction buffer (50% methanol/50% 0.5 M HCl) was added to tissue powder followed by two cycles of sonication, centrifugation, and supernatant collection. Tissue homogenate supernatant was pooled and frozen on dry ice then stored at −80° C. until drug analysis. The extraction efficacy of this methodology was assumed to be 100% for calculation purposes.

### Drug Analysis

Plasma samples and eye tissue extracts were analyzed for pazopanib using a validated analytical method based on protein precipitation, followed by HPLC/MS/MS analysis, Using 50 μl aliquot of plasma and eye tissue extract, the lower limit of quantification of pazopanib was 1 ng/ml for plasma and 10 ng/ml for eye tissue extract. The higher limit of quantification was 500 ng/ml for plasma and 5000 ng/ml for eye tissue extracts. The computer systems that were used on these studies to acquire and quantify data included Analyst Version 1.4,1 and SMS2000 Version 1.6. Plasma sample concentrations were expressed as ng pazopanib/ml. Tissue concentrations were determined by the following formula: Pazopanib ng/g tissue=([concentration in supernatant ng/ml*extract volume ml]/tissue weight g).

### Statistical Analysis

Results are expressed as means±standard deviation (SD) if not indicated otherwise. Statistical comparisons were performed using ANOVA and significant differences were judged at p<0.05 to reject the null hypothesis.

### Results

### Pazopanib Suppresses Development of CNV in a Rat Model of CNV

To determine whether pazopanib affects experimental CNV in vivo neovascularisation was induced in eyes of rats by subjecting the Bruch's membrane to a laser-induced rupture. This methodology has been commonly applied in experimental studies of neovascular AMD and allows predictions to be made on drug efficacy in humans. In particular, VEGF expression becomes upregulated and effects of VEGFR as well as PDGFR tyrosine kinase receptors can be well predicted since such like antagonists inhibit CNV. See, Yi X, Ogata N, Komada M, Yamamoto C, Takahashi K, Omori K, Uyama M. Vascular endothelial growth factor expression in choroidal neovascularization in rats. Graefe's Arch Clin Exp Ophthalmol. 1997;235:313-319; Shen W Y, Yu M J, Barry C J, Constable I J, Rakoczy P E. Expression of cell adhesion molecules and vascular endothelial growth factor in experimental choroidal neovascularisation in the rat. Br J Ophthalmol. 1998;82:1063-1071; Kwak N, Okamoto N, Wood J M, Campochiaro P A. VEGF is major stimulator in model of choroidal neovascularization. Invest Ophthalmol Vis Sci. 2000;41:3158-3164.

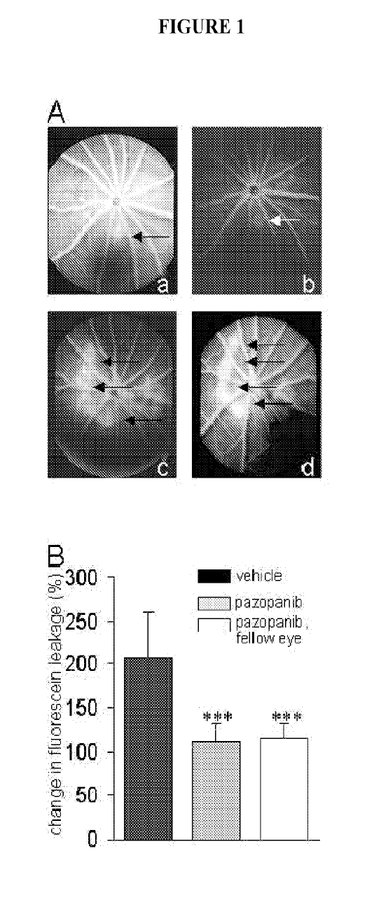

When areas of vessel leakage were followed up by fluorescence angiography from post laser days 7 to 14, topically administered pazopanib significantly reduced development of CNV lesions. In contrast, leakage of CNV lesions continued to progress in eyes of the control group treated with the vehicle (FIG. 1A; p<0.001), In FIG. 1A, panels a and c demonstrate leakage of fluorescein in the photocoagulated lesions seven days following laser injury. Topical application of pazopanib significantly reduced the progression of CNV leakage by postlaser day 14 (represented by panels b), compared to eyes of the vehicle control group (represented by panels d and c). Sites of laser injury are indicated with arrows.

Specifically, when the eyes were treated with the drug, the area of fluorescein leakage revealed non-significant changes to 111.41±21.34% (mean±SD, baseline lesion size normalized to 100% at day 7), whereas control eyes developed an increase up to 208.5±51.51% (FIG. 1B). These results indicated that a twice-daily topical administration of pazopanib inhibited further lesion development by >89%. Remarkably, applying pazopanib topically to the fellow (contralateral) eye also significantly inhibited lesion size progression in these studies. Thus, in lesioned eyes whose fellow eye was pazopanib-treated, CNV demonstrated only a marginal increase to 115.24±16.72% of baseline (FIG. 1B).

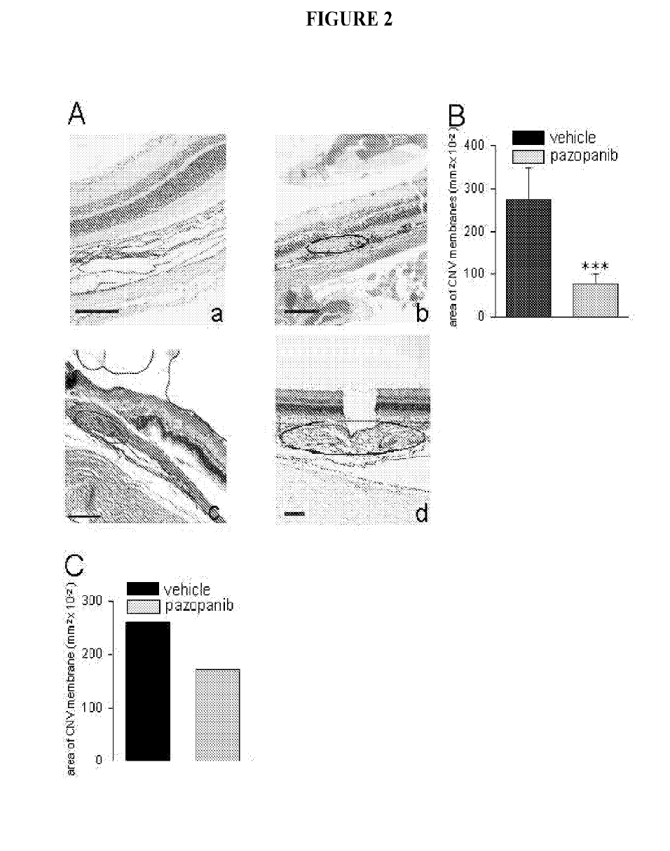

Additionally, histological retinal sections were analyzed on day 14 after laser treatment using staining with hematoxylin and eosin or immunohistochemistry. FIGS. 2A and B demonstrate that CNV lesions in vehicle-treated eyes (FIG. 2A, panels b and c) were larger than those treated topically with pazopanib. FIG. 2A shows choroid/ retinal sections derived from eyes without laser treatment (a) and from the lasered eyes (b-d) which either were treated topically with pazopanib (b) or vehicle (d: control group). Note reduction of CNV lesions when the contralateral eye was treated (c). Assessing the extent of CNV by measuring the relative thickness of the CNV membrane in the lesions revealed a significant difference. While the lesion area in vehicle-treated eyes was 27,397.3±7,386.4 μm² the area in eyes treated with pazopanib amounted to 7,760.3±2,312.0 μm². Thus a significant 71.7% inhibition in lesion size compared to vehicle control (p<0.001) (FIG. 2B) was noted. Neovascular areas were measured by quantitative digital image analysis. In another study, lasered eyes from rats treated topically with pazopanib in the fellow eye demonstrated a ˜34% inhibition in lesion size (FIG. 2C). The histology data together with the data obtained by fluorescence angiography (see above) point to a systemic effect produced by the drug in the fellow eye, which implies that a low oral dose that is able to achieve a similar systemic effect should be effective in treating CNV and, thus, disorders of ocular angiogenesis or vascular leakage such as those described above.

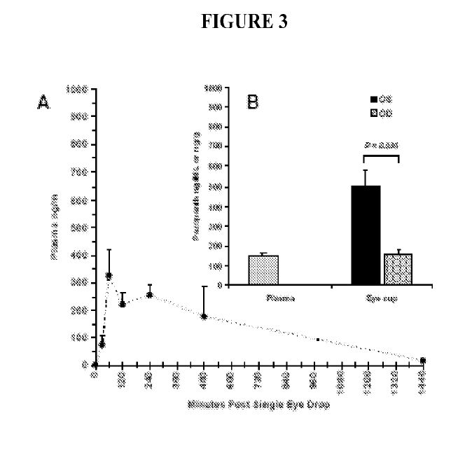

Topical administration of pazopanib results in detectable drug in the plasma of Norway Brown rats. After administration of a single 30 μl eye drop, peak levels of pazopanib were measured 60 minutes later in the 300 ng/ml range (FIG. 3A). Plasma levels declined to undetectable levels after 24 hours. Topical administration of single 30 μl drops to the left eye (OS) only, three times over a 24 hour period, resulted in a mean of 503 ng/g pazopanib in the treated eye cup tissues (sclera, choroid, and retina). Interestingly, detectable levels were also observed in the fellow (OD) eye (mean=159 ng/g) although at a statistically lower amount (FIG. 3B).

### Examination of Occular Tissue Distribution and Systemic Concentrations of Pazopanib

The ocular tissue distribution and systemic concentrations of radioactivity were assessed following topical ocular administration of pazopanib to Dutch belted (pigmented) rabbits. A single 60-μL at a target dose of 0.3 mg/dose (30 μCi/dose) was administered to the right eye. Blood and ocular tissues from both the dosed and non-dosed (left) eye were collected from one animal at each of eight sampling times up to 24 h, and analyzed for total radioactivity.

The levels of radioactivity were quantifiable in all blood and plasma samples from earliest sampling time (0.25 h) to the last sampling time (24 h) with the highest concentrations observed at 1 h in blood (11.3 ng eq/g) and at 2 h in plasma (12,8 ng eq/g).

Choroid levels reached (40.6 ng eq/g) at 2 h and peaked at (60.1 ng eq/g) at 8 h. Similarly retina levels reached (9.17 ng eq/g) at 2 h and peaked at (10.2 ng eq/g) at 4 h. This shows that more than half of the maximum levels in the CNV target tissues were reached within the first 2 hours after eye drop administration. Based on Stokes Einstein equation for the diffusion of a small molecule in water, it is estimated that in the best case scenario pazopanib would have diffused via local drug diffusion at most 0.4 cm in 2 hours which is much less than the size of the rabbit eye (approximately 1-2 cm). Without a systemic contribution, local drug diffusion could not explain the speed at which pazopanib reached the retina and the choroid.

The results obtained in this study provide evidence that the systemic delivery route provides an important contribution to the retina and choroid tissue levels. In addition it shows that any efficacy seen in the untreated eye would come mainly from low level systemically delivered drug.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method of treating a disorder of ocular angiogenesis or vascular leakage in a patient suffering from such condition comprising orally administering to the patient between 1 and 50 mg of a compound of formula (I): 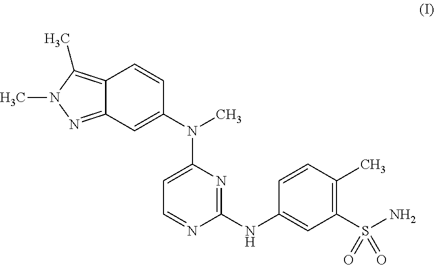 [SMILES: Cc1ccc(Nc2nccc(N(C)c3ccc4c(C)n(C)nc4c3)n2)cc1S(N)(=O)=O]
or a pharmaceutically acceptable salt or hydrate thereof.

2. The method according to claim 1, comprising administering between 1 and 20 mg of a compound of formula (I) or a pharmaceutically acceptable salt or hydrate thereof.

3. The method according to claim 1, comprising administering between 5 and 15 mg of a compound of formula (I) or a pharmaceutically acceptable salt or hydrate thereof.

4. The method according to claim 1 wherein the compound is a compound of formula (I′): 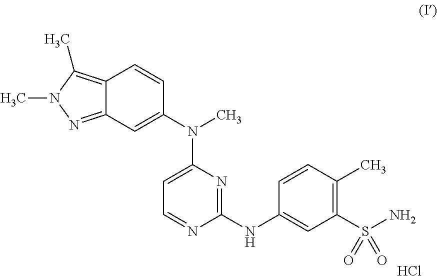 [SMILES: Cc1ccc(Nc2nccc(N(C)c3ccc4c(C)n(C)nc4c3)n2)cc1S(N)(=O)=O ; Cl]
or a hydrate thereof.

5. The method according to claim 1, wherein the compound is a compound of formula (I″):

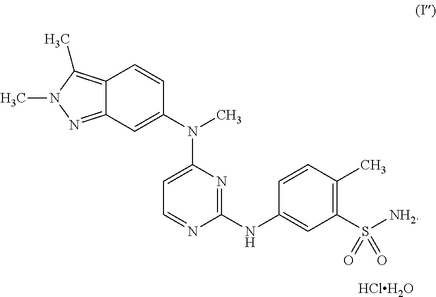

6. A method of treating neovascular age-related macular degeneration in a patient suffering from such condition comprising orally administering to the patient between 1 and 50 mg of a compound of formula (I): 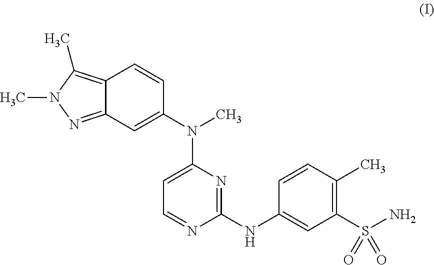 [SMILES: Cc1ccc(Nc2nccc(N(C)c3ccc4c(C)n(C)nc4c3)n2)cc1S(N)(=O)=O]
or a pharmaceutically acceptable salt or hydrate thereof

7. The method according to claim 6, comprising administering between 1 and 20 mg of a compound of formula (I) or a pharmaceutically acceptable salt or hydrate thereof.

8. The method according to claim 6, comprising administering between 5 and 15 mg of a compound of formula (I) or a pharmaceutically acceptable salt or hydrate thereof.

9. The method according to claim 6, wherein the neovascular age-related macular degeneration is wet age-related macular degeneration.

10. The method according to claim 6, wherein the neovascular age-related macular degeneration is dry age-related macular degeneration and the patient is characterized as being at increased risk of developing wet age-related macular degeneration.

11. The method according to claim 6, wherein the compound is a compound of formula (I′): 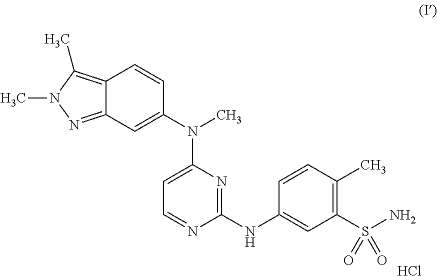 [SMILES: Cc1ccc(Nc2nccc(N(C)c3ccc4c(C)n(C)nc4c3)n2)cc1S(N)(=O)=O ; Cl]
or a hydrate thereof.

12. The method according to claim 6 wherein the compound is a complex of formula (I″):

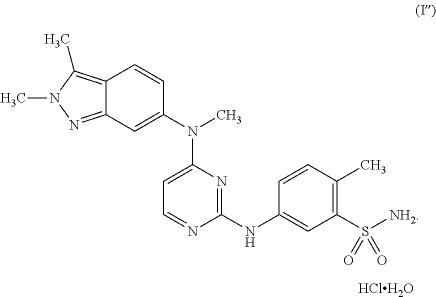

13. A method of treating a disorder of ocular angiogenesis or vascular leakage in a patient suffering from such condition comprising orally administering to the patient between 1 and 50 mg of a compound of formula (II): 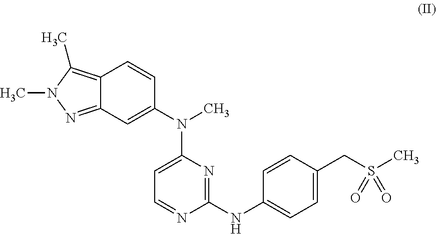
or a pharmaceutically acceptable salt thereof.

14. The method according to claim 13, comprising administering between 1 and 20 mg of a compound of formula (I) or a pharmaceutically acceptable salt or hydrate thereof.

15. The method according to claim 13, comprising administering between 5 and 15 mg of a compound of formula (I) or a pharmaceutically acceptable salt or hydrate thereof.

16. A method of treating neovascular age-related macular degeneration in a patient suffering from such condition comprising orally administering to the patient between 1 and 50 mg of a compound of formula (II): 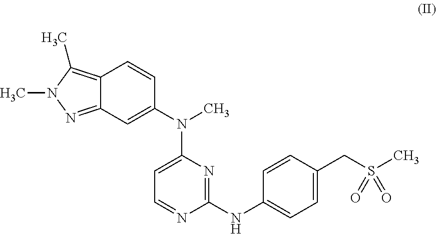
or a pharmaceutically acceptable salt thereof.

17. The method according to claim 16, comprising administering between 1 and 20 mg of a compound of formula (I) or a pharmaceutically acceptable salt or hydrate thereof.

18. The method according to claim 16, comprising administering between 5 and 15 mg of a compound of formula (I) or a pharmaceutically acceptable salt or hydrate thereof.

19. The method according to claim 16, wherein the neovascular age-related macular degeneration is wet age-related macular degeneration.

20. The method according to claim 16 wherein the neovascular age-related macular degeneration is dry age-related macular
